(19) United States
(12) Patent Application Publication
(10) Pub. No.: US 2010/0298761 A1
(43) Pub. Date: Nov. 25, 2010

(54) ELECTROPORATION DEVICE WITH IMPROVED TIP AND ELECTRODE SUPPORT

(75) Inventors: Lasse Guldborg Staal, Jyllinge (DK); Rune Henri Wincenty Drage, Greve (DK); René Larsen, Sandved (DK)

Correspondence Address: NIXON PEABODY, LLP, 300 S. Riverside Plaza, 16th Floor, CHICAGO, IL, 60606-6613, US

(73) Assignee: Sonion A/S, Roskilde (DK)

(21) Appl. No.: 12/782,454

(22) Filed: May 18, 2010

Related U.S. Application Data
(60) Provisional application No. 61/179,960, filed on May 20, 2009.

(51) Int. Cl. A61N 1/30

(52) U.S. Cl. 604/20

(57) ABSTRACT

An electroporation device having a handle section, a set of electrodes, and an electrode guide assembly. The handle section is operatively connected to an elongate introducer shaft, which has a longitudinal axis and a distal tip. The set of electrodes includes respective distal electrode tips. The electrode guide assembly is arranged inside the elongate introducer shaft and includes a set of electrode channels with each electrode channel guiding one or several electrodes. Each electrode is slidably arranged within an electrode channel between (a) a retracted position, where the distal electrode tips are enclosed inside the introducer shaft, and (b) an advanced position, where the distal electrode tips project to an outside of the introducer shaft. A set of longitudinally extending tip electrode channels are arranged in the distal tip and adapted to guide respective travel trajectories of the distal electrode tips during electrode advancement and retraction. The set of longitudinally extending tip electrode channels includes a set of electrode grooves disposed around a circumferential contour of a rotationally symmetrical member of the distal tip.

### CROSS-REFERENCE TO RELATED APPLICATION

This application claims the benefit of U.S. Provisional Application Ser. No. 61/179,960, filed May 20, 2009, entitled “Electroporation device with Improved Tip and Electrode Support.”

### COPYRIGHT

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves all copyright rights whatsoever.

### FIELD OF THE INVENTION

The present invention relates to an electroporation device with a distal tip which comprises a rotationally symmetrical member. A set of longitudinally extending tip electrode channels comprises a set of electrode grooves disposed around a circumferential contour of the rotationally symmetrical member. According to another aspect of the present invention there is provided an electroporation device with a first and a second set of electrode tubes forming a plurality of telescopic arrangements to support and protect electroporation electrodes. Electroporation devices in accordance with the present inventions are particularly well-suited for performing electroporation in deeper-lying bodily tissues for example tissues of the central nervous system such as brain tissue.

### BACKGROUND OF THE INVENTION

In the treatment of diseases in the brain, e.g. brain cancer, as well as diseases in other anatomical areas of a body, physical access to a diseased tissue region may be a challenge. This is especially the case if the diseased region lies deep within the body of the patient. Furthermore, efficient delivery and subsequent uptake of therapeutic molecules, such as a drug or genetic compound, to an anatomical target tissue often presents a significant challenge.

Electroporation is a known method used to deliver drugs and genetic compounds and other substances to various types of biological target tissues, where the uptake of these substances into tissue cells is enhanced through the application of electric pulses of specific amplitudes. The delivery of chemotherapeutic agents by electroporation is also known as Electro-Chemotherapy (ECT) and the delivery of genes as Electro Gene Transfer (EGT). In ECT and EGT applications, electroporation is used to create a transient permeabilization of the cell membranes in a target tissue area for the purpose of enhancing uptake of the chemotherapeutic agents as well as the uptake and expression of genetic materials.

Electroporation is a science of thresholds, and the ultimate goal is to precisely and reliably generate an electrical field that may promote uniform uptake of biologically active molecules in a target tissue. In this process, burning/scarring of target and/or adjacent tissue is strongly undesirable, since it may interfere with the uptake of molecules through changes in tissue conductivity.

In order to provide efficient electroporation one or several pair(s) of electrode poles has to be brought into—or into close vicinity of—the region to be treated (target region). WO 2007/144004 A1 discloses a prior art electroporation device which comprises an elongate introducer shaft with a rounded distal tip for insertion into a patient's body. The rounded tip seeks to minimize displacement-related trauma to intervening tissue through which the shaft need to project to reach the target tissue or treatment region. A set of electrodes can be slidably advanced through a number guide channels and tip electrode channels to contact the target tissue once the rounded distal tip has been appropriately positioned in the patient's body. The distal tip portion of the disclosed electroporation device is composed of eight identical wedge-shaped individual tip elements or members arranged around a central longitudinal axis of the introducer shaft. Each wedge-shaped member has a distal rounded portion, a plane triangular proximal surface and two 45 degrees angled plane faces or surfaces oriented in direction of the central longitudinal axis. Grooves are engraved into one of the plane faces of each wedge-shaped member. A tapering outer contour of the tip is provided by abutting the 45 degrees angled faces of the eight wedge-shaped members around the central longitudinal axis. The spatial configuration of the tip electrode channels defined by the abutted wedge-shaped tip elements would require a set of alignment bushings to compensate for a difference in spatial positions of the electrode guide channels inside the introducer shaft and the tip electrode channels located in the rounded distal tip.

Another potential problem associated with the structure of the disclosed multi-element tip is the risk of tip element detachment of the wedge-shaped elements during insertion of the electroporation device. A thinned proximal portion of the distal tip is inserted into an inner lumen of tubular introducer shaft to retain each of the wedge-shaped members in a fixed position. However, the wedge-shaped contour and orientation of the individual wedge-shaped members or tip elements make these prone to sliding out or drifting apart along the central longitudinal axis of the introducer shaft without an adequate locking mechanism coupled to the tip elements.

Tip element detachment constitutes a potential patient safety hazard because the fork-shaped contour that will result if the wedge-shaped members slide apart.

The electroporation device disclosed in WO 2007/144004 A1 furthermore comprises an adaptor plate attached to proximal end portions of the electrodes. The adaptor plate serves as an electrical connector between the electrodes and a remote electrical applicator which generates electrical electroporation pulses. The adaptor plate is fixedly connected to a user operable deployment mechanism to allow the user to displace the electrodes between a retracted position and an extended position. A number of cylindrical guide sheaths which each enclose a set of 4 mutually isolated and parellelly extending electrodes are enclosed inside the elongate introducer shaft. The disclosed configuration of electrodes with surrounding cylindrical guide sheaths lack a mechanism to guide and support sliding movement of the individual electrodes in longitudinal direction so as to prevent electrode bending or buckling during electrode advancement. This problem is particularly pronounced for electroporation devices with small dimensions which imply severe size constraints of the electrodes making these vulnerable to bucking or bending forces during electrode advancement.

### SUMMARY OF THE INVENTION

According to a first aspect of the invention there is provided an electroporation device comprising a handle section operatively connected to an elongate introducer shaft which comprises a longitudinal axis and a distal tip. The electroporation device comprises a set of electrodes comprising respective distal electrode tips and an electrode guide assembly arranged inside the elongate introducer shaft. The electrode guide assembly comprises a set of electrode channels with each electrode channel guiding one or several electrodes. Each of the electrodes is slidably arranged within an electrode channel between a retracted position, where the distal electrode tips are enclosed inside the introducer shaft, and an advanced position, where the distal electrode tips project to an outside of the introducer shaft. A set of longitudinally extending tip electrode channels is arranged in the distal tip and adapted to guide respective travel trajectories of the distal electrode tips during electrode advancement and retraction. The set of longitudinally extending tip electrode channels comprises a set of electrode grooves disposed around a circumferential contour of a rotationally symmetrical member of the distal tip.

In accordance with the present invention, the rotationally symmetrical member of the distal tip simplifies miniature assembly and effective space utilization of an inner lumen of the elongate introducer shaft. These are important advantages because the outer dimensions of an electroporation device, in particular invasive portions thereof, are generally made a small as practically feasible to minimize tissue injury during advancement of the elongate introducer shaft through patient tissue. The set of electrode channels may be configured as a compact annularly shaped array of electrodes channels, or even several coaxially-arranged annularly shaped sets electrode channels, where spatial positions of individual electrode channels are aligned with corresponding spatial positions of the tip electrode channels so as to make alignment bushings or similar electrode redirection members superfluous.

Electroporation devices in accordance with the present invention may used to create irreversible electroporation effects in the target tissue by an appropriate choice of electric field strength. Alternatively, the present electroporation device may used to create transient permeabilization of cell membranes of the target tissue in connection with introducing particular therapeutic molecules or substance(s) into cells of the target tissue.

The handle section of the present an electroporation device may in certain embodiments of the invention be shaped and sized to be hand-hand held to facilitate manipulation by a surgeon or a doctor responsible for an electroporation procedure. In other embodiments of the invention, the handle section may be shaped and sized for attachment to an appropriately shaped holder of a piece of stereo tactical equipment or a similar support structure without necessarily involving direct human contact.

The elongate introducer shaft may in some embodiments of the invention be formed as an essentially rigid non-bendable structure. Alternatively, the elongate introducer shaft may be formed as a soft flexible cylindrical structure for example resembling a catheter or an endoscope with sufficient bendability to navigate past certain tissue structures using natural body lumens as pathways.

The distal tip may be detachable from the elongate introducer shaft to provide a modular electroporation device based on disposable or single use pre-sterilized parts. Since the outermost surface of the distal tip necessarily must be brought into contact with the patient's tissue during treatment, there should exist a practical manner to either sterilize or replace the distal tip before each treatment session. Alternatively the distal tip may be formed integrally with, or bonded to, a disposable elongate introducer shaft which comprises an appropriate detachable locking mechanism to the handle section.

The electrode guide assembly may be configured with various shapes and dimensions conforming to an inner wall geometry of the elongate handle section. The electrode guide assembly may comprise one or several plates, discs or annular structures with through going apertures or holes configured to receive and control positions and spacing of the electrode channels. In other embodiments, the electrode guide assembly comprises an elongate essentially solid body extending throughout a major portion of an inner lumen of the elongate introducer shaft with the electrode channels designed as longitudinally oriented straight closed, semi-open or open channels or grooves formed or engraved into an outer surface of the electrode guide assembly.

Each of the electrodes may have a substantially circular cross-sectional profile with a diameter of less than 2 mm, preferably less than 1 mm, more preferably less than 0.5 mm, and even more preferably less than 0.20 mm. The distal electrode tips or electrically conductive electrode ends are electrically conductive segments of the electrodes. Each of the distal electrode tips preferably comprises an electrically un-insulated or conductive portion with a length between 10 mm and 50 mm. The distal electrode tips may have substantially identical cross-sectional profiles and dimensions to those of the electrodes themselves. Alternatively, the distal electrode tips may have a different cross-sectional profile and/or larger dimensions. The distal electrode tips may for example have spherical shape with a larger radius than the maximum cross-sectional dimension of an associated electrode.

In a particularly advantageous embodiment of the invention, the set of electrode grooves is formed in a single unitary rotationally symmetrical member. All electrode channels of the set of electrode channels are accordingly formed in a single member reducing the risk of detachment of individual tip parts during patient treatment. Cleaning and sterilization is facilitated through the use of the single unitary member that does not comprise abutting joined surfaces where contaminating substances may accumulate. Likewise, tip manufacturing and assembly procedures can be conducted in a fast and economical manner without manufacturing steps related to the assembly and joining of a multitude of irregularly shaped miniature items.

The single unitary rotationally symmetrical member may be fabricated by injection moulding of a suitable resin such as thermoplastic elastomers PPSU, PSU and PE 1000 which are amorphous resins combining excellent thermal stability and good resistance to environmental stress cracking. However, other biocompatible thermoplastic elastomers or electrically inert metals (e.g. anodized aluminium), ceramics and other shapeable materials that are compatible with sterile autoclaving processing may alternatively be used. The set of grooves, which form part of the longitudinally extending tip electrode channels, may be formed during the injection moulding process through a suitable mould design and mould inserts. In other embodiments of the invention, the set of electrode grooves are formed in the circumferential contour of the single unitary rotationally symmetrical member by material removing processes such as chipping, machining, lathing, spark erosion, laser ablation, milling, bark eroding, etching etc. In one variant of the single unitary rotationally symmetrical member, the set of electrode grooves comprises at least two grooves arranged at different depths in the circumferential contour of the member. According to this embodiment, electrode grooves at two or more different depths may be located around the circumferential contour in an alternating pattern. For example, every second electrode groove may be placed shallowly below circumferential contour while the intermediate electrode grooves are engraved to a deeper lying position toward a central axis of the single unitary and rotationally symmetrical distal tip. The alternating pattern of shallow and deeper lying electrode grooves allows the corresponding longitudinally extending tip electrode channels to guide the travel trajectories of the distal electrode tips, or terminal tips, during electrode advancement so that a predetermined three-dimensional electrode distribution pattern is created at or surrounding the target tissue.

According to another embodiment of the invention, the distal tip comprises a plurality of coaxially arranged rotationally symmetrical members. The set of longitudinally extending tip electrode channels being formed as respective sets of electrode grooves engraved into respective circumferential contours of the plurality of coaxially arranged rotationally symmetrical members. This embodiment may provide a space efficient distribution of the electrodes with two or more co-axially arranged annular patterns of tip electrode channels centred around the longitudinal axis of the elongate introducer shaft. In one embodiment, the distal tip comprises a pair of coaxially arranged hollow shells in form of an inner, rotationally symmetrical, shell and an outer shell. The set of longitudinally extending tip electrode channels are formed by the set of electrode grooves engraved into a circumferential outer wall of the inner hollow shell and inner wall sections of the outer hollow shell. In this embodiment, closed electrode channels (in a direction perpendicular to the longitudinal axis), are formed by cooperating walls of the inner and outer hollow shells.

The distal tip is preferably configured with an outermost surface or contour that is substantially smooth, rounded, and non-cutting with a substantially smooth, non-cutting transition to the elongate introducer shaft. This is true both for distal tip designs where all electrode channels are formed in a single unitary rotationally symmetrical member and for designs based on a plurality of coaxially arranged rotationally symmetrical members. The smooth, rounded, and non-cutting tip contour allows the elongate introducer shaft to be advanced through the patient's body to the target tissue in a manner that causes minimal trauma to intervening tissue by spreading the intervening tissue instead of cutting through it. This feature is naturally particularly advantageous for applications in brain tissue.

The rotationally symmetrical member preferably has n-fold rotational symmetry where n is a positive integer number between 2 and 32 for example between 4 and 16, such as between 6 and 12. In case the set of tip electrode channels is formed in single unitary rotationally symmetrical member, it may be preferable to provide a large number of electrode grooves around its circumferential outer contour such as between 12 and 32. If the distal tip on the other hand comprises a plurality of coaxially arranged rotationally symmetrical members, it may be advantageous to place a smaller number of electrode grooves around the circumferential contour of each member due to the typical smaller dimensions of each of the coaxially arranged members compared to the dimensions of the single unitary member. In this case, each of the coaxially arranged members may carry between 3 and 9 electrode grooves so that the entire distal tip comprises a number of electrode grooves equaling 3 to 9 times the number of coaxially arranged rotationally symmetrical members of the distal tip. Due to the coaxial arrangement of the rotationally symmetrical members, the resulting electrode channels are automatically formed in sets placed on different radial coordinates of the distal tip.

The distal electrode tips are preferably adapted to extend beyond the distal tip in the advanced position of the set of electrodes to allow the distal electrode tips to be placed nearby or enclose the target tissue without penetration thereof by the elongate introducer shaft. In other situations, one or several of the distal electrode tips may penetrate the target tissue to reach distal positions situated behind the target tissue in the patient's body.

To create a treatment region of larger dimensions than a cross-section of the elongate introducer shaft itself, the present electroporation device may advantageously comprise a mechanism for deflecting the distal electrode tips away from the longitudinal axis of the elongate introducer shaft during electrode advancement to their advanced position such that at least one planar projection taken in a plane perpendicular to said longitudinal axis of a distance between at least one pair of electrode tips is larger than a maximum dimension of the cross-section of the elongate introducer shaft; The cross-section taken in a plane perpendicular to said longitudinal axis at a distal end of the elongate introducer shaft.

In a preferred embodiment, the deflection of each of the pair of distal electrode tips is provided by a corresponding curvature of the longitudinally extending tip electrode channel. The curvature of each of the longitudinally extending tip electrode channel is preferably between 1 and 45 degrees relative to the direction of the longitudinal axis. Furthermore, in accordance with another preferred embodiment, the respective curvatures of the longitudinally extending tip electrode channels constitute the sole mechanism for determining the travel trajectories of the distal electrode tips during electrode advancement. This latter feature simplifies the construction of the electrodes and an associated deployment mechanism adapted to displace the electrodes between the retracted and advanced positions. The electrodes may be fabricated without resorting to shape memory alloys or pretension mechanisms.

According to an advantageous embodiment of the invention, each electrode channel of the set of electrode channels in the electrode guide assembly is aligned with a corresponding longitudinally extending tip electrode channel of the distal tip. This embodiment makes it possible to skip intermediately located re-directing or alignment bushings of prior art devices that align the tip electrode channels to the electrode channels of the elongate introducer shaft. The alignment of the electrode channels and the longitudinally extending tip electrode channels provided in this embodiment of the invention allows the number of individual parts of the electroporation device to be reduced leading to reduced parts costs, tooling costs and assembly costs. Furthermore, a reduction of frictional resistance associated electrode movement through a transition region between the electrode guide assembly and the distal tip improves the quality of tactile feedback delivered to the operator/surgeon by the electroporation device during electrode advancement.

In an advantageous embodiment, each of the electrode channels comprises a metallic or plastic cannula tube, of a first set of cannula tubes, surrounding or enclosing a portion of an electrode in the electrode channel. The metallic or plastic cannula tube serves the purpose of electrically insulating each of the electrodes and/or provides mechanical support to prevent the previously described electrode bending or buckling during electrode advancement. The first set of cannula tubes are preferably fixedly attached to the electrode guide assembly and therefore stationary relative to the elongate introducer shaft so that an electrode moves slidingly relative to the cannula tube during electrode advancement and retraction with sufficient space to allow unhindered movement of the electrode without scraping an inner wall of the cannula tube. To secure this unhindered sliding motion a difference in outer diameter of the electrode and an inner diameter of the cannula tube is preferably set to value between 0.1 mm and 0.6 mm such as between 0.2 and 0.4 mm.

In a particularly advantageous embodiment, the electroporation device further comprises a second set of cannula tubes which forms a plurality of telescopic arrangements with the first set of cannula tubes. According to this embodiment, the electroporation device comprises a deployment mechanism operatively connected to the electrodes through an adaptor plate to displace the set of electrodes between the retracted and advanced positions. A second set of cannula tubes are in addition fixedly attached to the adaptor plate and configured to form the plurality of telescopic arrangements with the first set of cannula tubes. The second set of cannula tubes is, by virtue of its fixed mounting to the adaptor plate, moveable along the longitudinal axis relative to the first set of cannula tubes. An overlap or overlap range between each of the telescopic cannula tube arrangements ensures the proximal portion of each electrode is surrounded by, or enclosed inside, a guiding structure even when the adaptor plate, and thereby the set of electrodes, is retracted. Without the second set of cannula tubes the stationary arrangement of the first set of cannula tubes relative to the elongate introducer shaft would leave the proximal portions of the electrodes exposed without any a surrounding support structure with the deployment mechanism (and the set of electrodes) in its retracted position. An arrangement without the second set of cannula tubes would substantially increase the risk of electrode buckling and electroporation device malfunctions.

In an embodiment the elongate introducer shaft further comprises an access channel extending there through along the longitudinal axis, preferably coincident with a central longitudinal axis. The set of electrodes may comprise a tubular electrode mounted in the access channel. The access channel may in certain embodiments function as a fluid delivery channel through which a dose of therapeutic molecules can be administered to the target tissue. The fluid delivery channel preferably extends through a length of the introducer shaft and terminates through a tip electrode channel. The handle section may comprise a therapeutic molecule delivery system with a therapeutic molecule reservoir and actuating means for administering said therapeutic molecules through said fluid delivery channel.

A cross sectional profile of the elongate introducer shaft may be essentially circular, oval or polygonal for example shaped as a regular pentagon, hexagon or octagon. The maximum dimension of a cross section of the elongate introducer shaft will depend on the particular application but may be smaller than 30 mm, preferably smaller than 10 mm, or more preferably less than 5 mm.

In any of the above-described embodiments each electrode may be assigned an individual electric polarity such that the electric stimuli signals can be provided from and between individual electrodes. Thereby it is achieved that electrode pairs may communicate electrically with each other in order to provide a clearly defined and configurable electric field in the target tissue. Further, by employing individually assignable electrodes the operator may customize a shape of the electrical field in the target tissue to contours of a given lesion or target tissue.

In any of the above electroporation device embodiments, the retracted position of the electrodes may render the distal electrode tips enclosed within the longitudinally extending tip electrode channels or even further retracted to a position within the electrode channels of the elongate introducer shaft body.

A second aspect of the invention relates to an electroporation device, comprising a handle section operatively connected to an elongate introducer shaft where the elongate introducer shaft has a longitudinal axis and a detachable distal tip. The electroporation device furthermore comprises a set of electrodes comprising respective distal electrode tips and an electrode guide assembly arranged inside the elongate introducer shaft and comprising a set of electrode channels with each electrode channel guiding one or several electrodes. Each electrode is slidably arranged within an electrode channel between a retracted position, where the distal electrode tips are enclosed inside the introducer shaft, and an advanced position, where the distal electrode tips project to an outside of the introducer shaft. A set of longitudinally extending tip electrode channels are arranged in the detachable distal tip and adapted to guide respective travel trajectories of the distal electrode tips during electrode advancement and retraction. In accordance with this second aspect of the invention, the distal tip comprises a first locking member engaging a second locking member arranged on the elongate introducer shaft.

In one embodiment, the first locking member comprises an annular protrusion, rise or collar integrally formed with the distal tip and protruding from an outer surface contour thereof. The annular protrusion ensures the distal tip may be locked to the introducer shaft by snap-fitting or press-fitting against a mating cylindrical inner wall section of the elongate introducer shaft.

The distal tip may comprises a plurality of coaxially arranged rotationally symmetrical members such as between 2 and 5 symmetrical hollow shells, where the set of longitudinally extending tip electrode channels are formed as respective sets of electrode grooves engraved into respective circumferential contours of the plurality of coaxially arranged rotationally symmetrical members.

In one embodiment of the electroporation device the first locking member comprises a semi-cylindrical shell coupled to the distal tip. The semi-cylindrical shell comprises a radially projecting pin. A second locking member, arranged in a wall of the elongate introducer shaft, comprises an aperture or opening mating to the radially projecting pin. The semi-cylindrical shell may comprise a toothed distal edge with a plurality of individual teeth adapted to frictionally engage and lock to respective tip electrode channels of the set of longitudinally extending tip electrode channels of the detachable distal tip.

According to yet another embodiment, the set of longitudinally extending tip electrode channels are formed in a single rotationally symmetrical member of the distal tip.

The first locking member may in certain embodiments be integrally formed with the single rotationally symmetrical member of the distal tip while the first locking member in other embodiments may be formed in a separate tip member attached to the single rotationally symmetrical member.

It is understood that the above-described functionality and features of the first aspect of the present invention are applicable to the second aspect of the invention as well.

According to a third aspect of the invention there is provided an electroporation device, comprising a handle section operatively connected to an elongate introducer shaft which has a longitudinal axis and a distal tip. A set of electrodes is at least partly enclosed, and slidably arrange, in respective ones of a first set of electrode tubes and comprises respective distal electrode tips. A deployment mechanism operatively connected to the set of electrodes is configured to displace these between a retracted position, where the distal electrode tips are enclosed inside the introducer shaft, and an advanced position where the distal electrode tips project to an outside of the introducer shaft. An electrode guide assembly is arranged inside the elongate introducer shaft and comprises a set of electrode channels each guiding one or several electrode tubes of the first set of electrode tubes. A set of longitudinally extending tip electrode channels is arranged in the distal tip and adapted to guide respective travel trajectories of the distal electrode tips during electrode advancement and retraction. The deployment mechanism comprises an adaptor plate fixedly attached to the set of electrodes and to a second set of electrode tubes forming a configuration where the first and second sets of electrode tubes form a plurality of telescopic arrangements.

As previously-mentioned, the plurality of telescopic arrangements advantageously allow the proximal portions of the electrodes to be enclosed within and supported by the second set of electrode tubes even when the deployment mechanism (and the set of electrodes) is retracted. An overlap or overlap distance of each telescopic arrangement may be adapted to fit length dimensions of any particular electroporation device, in particular fit a difference in distance between the retracted and advanced positions of the electrodes. Preferably, the overlap range of each telescopic arrangement between the first and second set of electrode or cannula tubes is larger than 20 mm, such as between 30 mm and 100 mm for example around 60-70 mm, measured along the longitudinal axis in the advanced position of the set of electrodes. Preferably, the overlap of each telescopic arrangement between the first and second set of electrode tubes is larger than 2 mm, preferably larger than 5 mm, in the retracted position of the set of electrodes to ensure that individual electrodes are adequately supported in the displacement range of the set of electrodes.

According to a preferred embodiment, each electrode tube of the second set of electrode tubes is substantially coaxially arranged relative to a respective one of the first set of electrode tubes. Individual electrode tubes of the second set of electrode tubes may of course be arranged either as the inner or the outer electrode tube of each of telescopic arrangement. In certain telescopic arrangements several inner electrode tubes are positioned inside each outer electrode tube. This will of course require that the inner electrode tubes are electrically insulted from each other for example fabricated in an electrically insulating material. Alternatively, or additionally, may electrical insulation be provided by having each of the electrodes mounted inside the inner electrode tubes coated with an electrically insulating agent.

Preferably, each electrode tube of the first set of electrode tubes is arranged outside or inside a single electrode tube of the second set of electrode tubes. This one-to-one telescopic arrangement of individual electrode tubes of the first and second sets allows a close proximity between concentric wall sections and close conformance of the inner electrode tube to an outer surface contour of the electrode to provide a compact arrangement.

The adaptor plate may be constructed in anyone of a range of different materials suitable for holding electrical conductor patterns. One embodiment comprises a single layer or multi-layer printed circuit board while another embodiment comprises a ceramic carrier substrate. The adaptor plate may comprise a plurality of through-going mounting holes receiving respective electrodes and electrode tubes of the second set of electrode tubes. The respective proximal ends of the electrodes are preferably inserted into the through-going mounting holes. The proximal ends of the electrodes may be attached to the adaptor plate by various interconnection and attachment methodologies such press-fitting, crimping, welding or soldering. The same type of attachment methodologies may be utilized to join or connect the second set of electrode tubes to the adaptor plate.

Finally, it is understood that the individual features and functions of the electroporation device as described above according to the first aspect of the invention, may be integrated into the present the electroporation device. These individual features and functions include, but are not limited to, dimensions, structures and materials of the distal tip, the electrodes, the cannula or electrode tubes and the elongate handle.

### BRIEF DESCRIPTION OF THE DRAWINGS

In the following the invention will be described in further detail with reference to the drawings. The drawings show ways of implementing the present invention and are not to be construed as being limiting to other possible embodiments falling within the scope of the appended set of claims.

FIG. 1 is a first cross-sectional view of electroporation device according to a preferred embodiment of the invention,

FIG. 2 is a second cross-sectional view of the electroporation device depicted in FIG. 1,

FIG. 3 is a perspective view of a single unitary rotationally symmetrical member suitable as part of the distal tip of the electroporation device depicted in FIGS. 1 and 2,

FIG. 3a) is an illustration of a coupling between the single unitary rotationally symmetrical member depicted on FIG. 3 and a tubular introducer shaft,

FIG. 4 is a close-up view of a telescopic arrangement of first and second sets of electrode tubes of the electroporation device,

FIG. 5 shows a cross-sectional view of the electroporation device with a set of electrode tubes coupled to an adaptor plate,

FIG. 6 shows a cross-sectional view similar to FIG. 5 but with the adaptor plate depicted in cross-sectional view,

FIG. 7 is a close-up cross-sectional view of the adaptor plate with mounted connector shoes,

FIGS. 8a) and b) are two different views of the adaptor plate alone,

FIGS. 9 a) and b) are frontal and cross-sectional views, respectively, of a multi-element distal tip coupled to a distal portion of an elongate introducer shaft according to a second embodiment of the invention,

FIG. 10 illustrates the multi-element distal tip depicted on FIG. 9 in a partially disassembled state,

FIG. 11 a)-d) is a series of illustrations of construction details of the multi-element distal tip depicted on FIGS. 9 and 10; and

FIGS. 12 a) and b) are respective frontal and rear views of the multi-element distal tip depicted on FIGS. 9-11 with mounted electrodes with the electrodes positioned in an advanced state or position.

### DETAILED DESCRIPTION OF EMBODIMENTS OF THE INVENTION

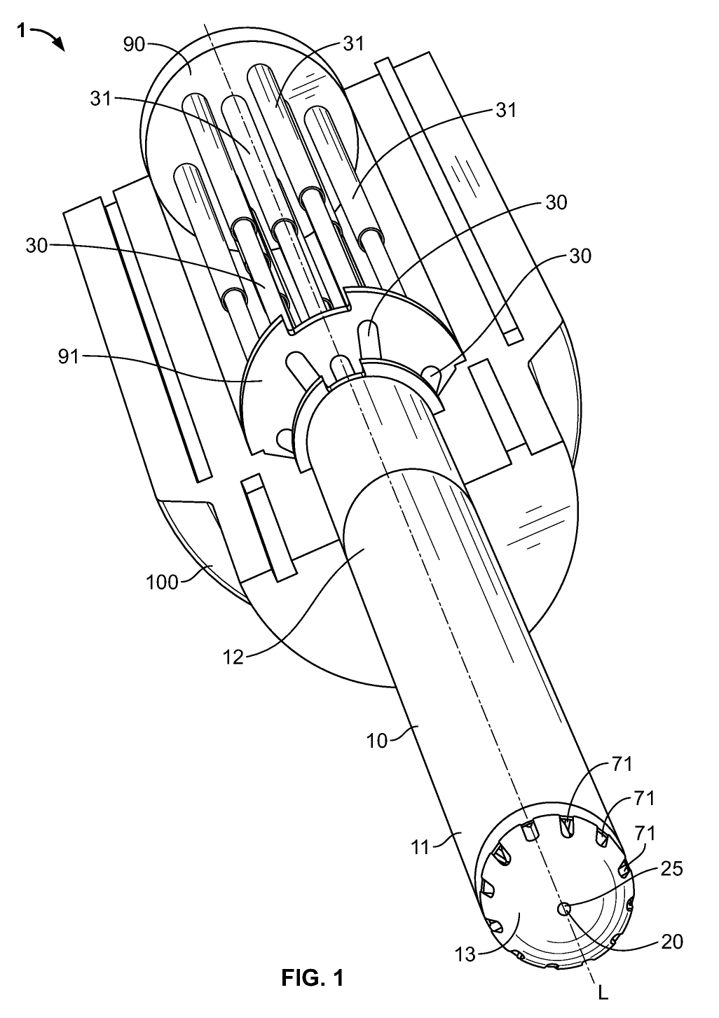

FIG. 1 illustrates an electroporation device 1 according to a preferred embodiment of the invention. The electroporation device 1 comprises a handle section 100 and an elongate and essentially tubular introducer shaft 10 or shaft preferably having a length suitable for accessing deeper-lying tissue regions. The length of the shaft 10 may be adapted for the intended use. The shaft 10 is attached to the handle section 100, and has a proximal end 12 adjacent to the handle section 100 and a distal end 11. The shaft 10 may in one embodiment be fixedly attached to the handle section. In other embodiments the shaft 10 may be detachably mounted to the handle section 100, and may comprise suitable connector mechanisms for establishing detachable electrical and mechanical interconnection, e.g. for conducting electrical pulses. A distal tip 13, that is preferably shaped to permit the creation of a channel through intervening layers of tissue while causing minimal damage to said tissue, is disposed at the distal end 11 of said shaft 10.

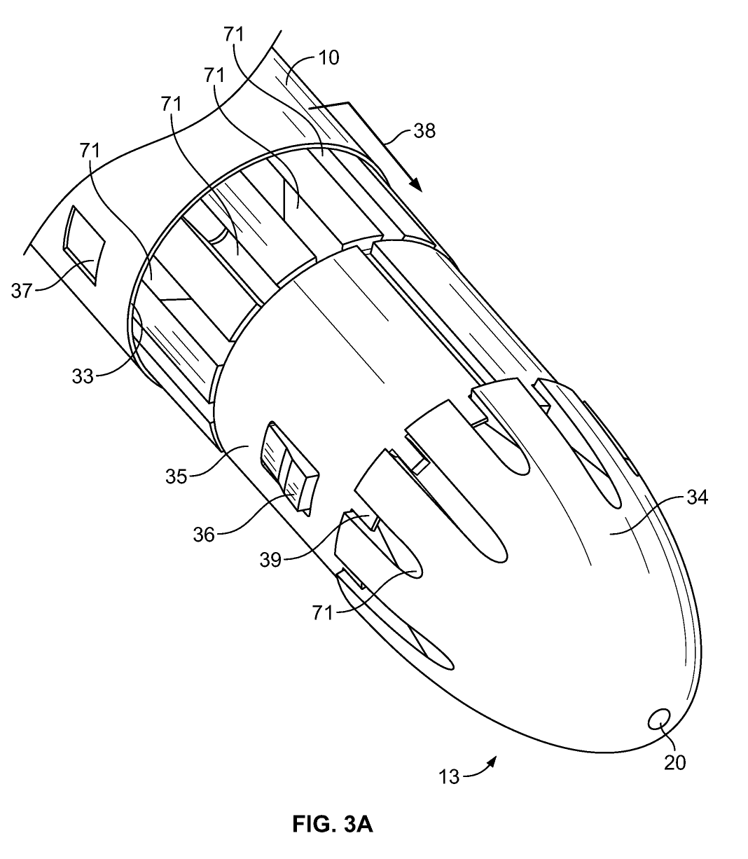

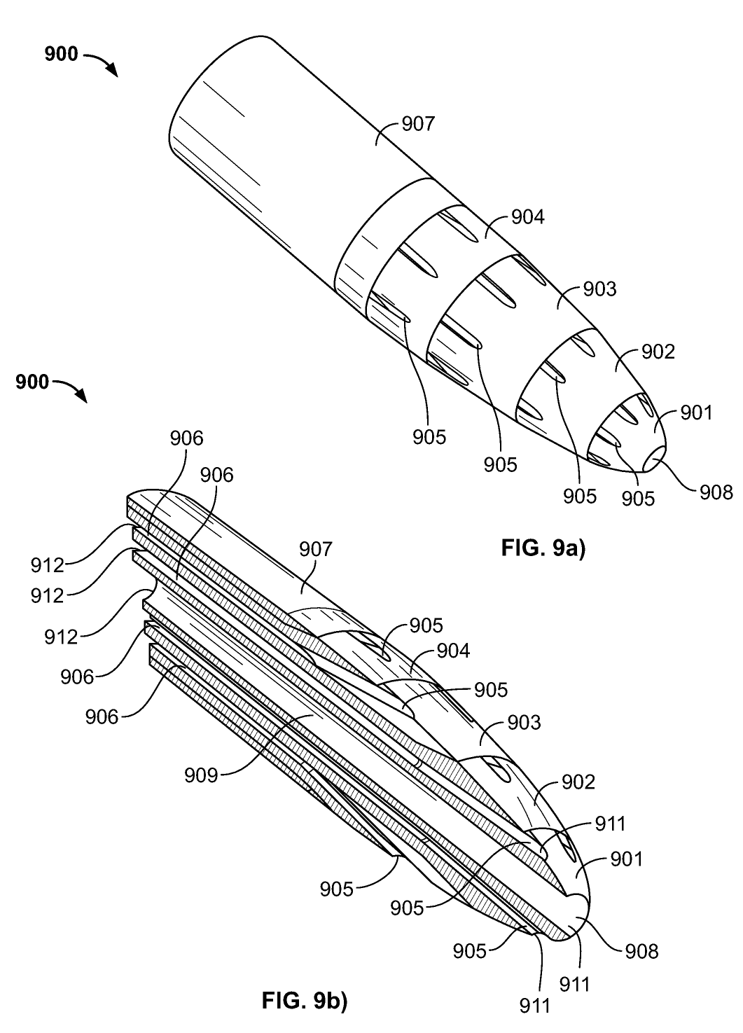

In the present embodiment of the invention, a set of tip electrode channels is formed in a single unitary and rotationally symmetrical member of the distal tip 13 as further discussed in connection with FIGS. 3 and 3a below. However, in other embodiments of the invention, the distal tip 13 may formed by a number of rotationally symmetrical and coaxially arranged members or hollow shells each comprising a set of tip electrode channels as explained in detail below in connection with a number of alternative distal tip designs disclosed in connection with the description of FIGS. 9-12.

The distal tip 13 has a rounded, non-cutting shape. In other embodiments (not shown) the distal tip 13 may be provided with a cutting edge or a pointed tip, i.e. a sharpened tip. These latter embodiments are particularly well-suited for percutaneous applications. In either case, the distal tip 13 may be formed integrally with the introducer shaft 10 or it may be a formed as a separate part attached or coupled to the distal end 11 of the introducer shaft 10 by a suitable locking mechanism. With a removable/detachable distal tip 13, and/or a detachable shaft 10, the length and thereby the reach of the electroporation device in treatment procedures, may be adapted to a specific treatment procedure, by a suitable choice of shaft 10. Further, this allows for use of only disposable parts for parts of the electroporation device 1 that are inserted into a patient. Thereby, the need for disinfection of the invasive parts of the electroporation device 1 may be eliminated.

The introducer shaft 10 comprises a centrally located fluid delivery channel 20 or access channel 20 extending through the shaft 10 from the proximal end 12 to the distal end 11 along a longitudinal axis, L, of said shaft 10. The access channel 20 terminates centrally through said distal tip 13 through a dedicated outlet 25. At the distal end 11 of the shaft 10 one or more outlets 25 of the delivery channel 20 are provided in the distal tip 13 in order to administer an amount of fluid/medical compound adjacent to the distal tip 13. In the embodiments shown in the figures a single outlet 25 is provided, however, the access channel 20 may split up into a multitude of minute channels at the distal end 11, each having an outlet at the distal tip 13. The access channel 20 extends through the shaft 10 to the handle section 100, and is adapted for connection to a drug/genetic material delivery means (not shown) comprising a storage of a drug/medicament and/or means (e.g. a pump or a piston or the like) for transporting said medicament from said storage and through the access channel 20 to a target tissue. In a simple form the delivery means may be provided by a syringe (not shown), connected to the delivery channel 20 via the handle section 100, e.g. by a suitable tubing.

The electroporation device 1 and the access channel 20 may also be configured by e.g. appropriate coupling means and/or dimensioning to receive and guide for instance an ultrasound probe, a surgical tool or another tool for minimally invasive manipulation of tissue. Thus the device 1 can be used in a flexible way, where for example it is not necessary to remove the device 1 and replace it with another specialized surgical tool, if the operator/surgeon encounters unexpected obstacles/difficulties prior to, during or following the electroporation treatment.

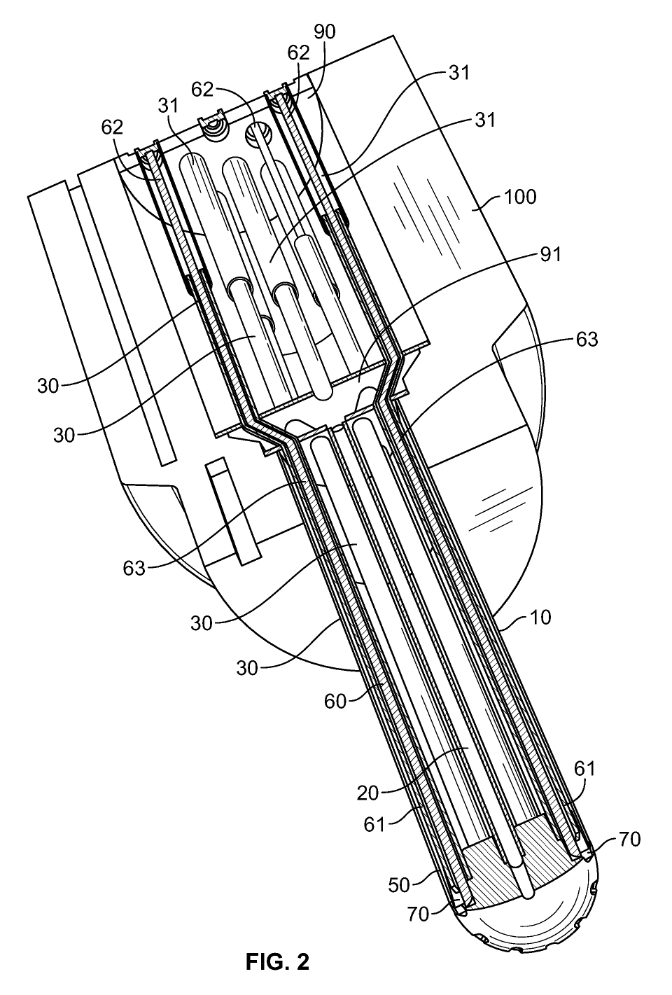

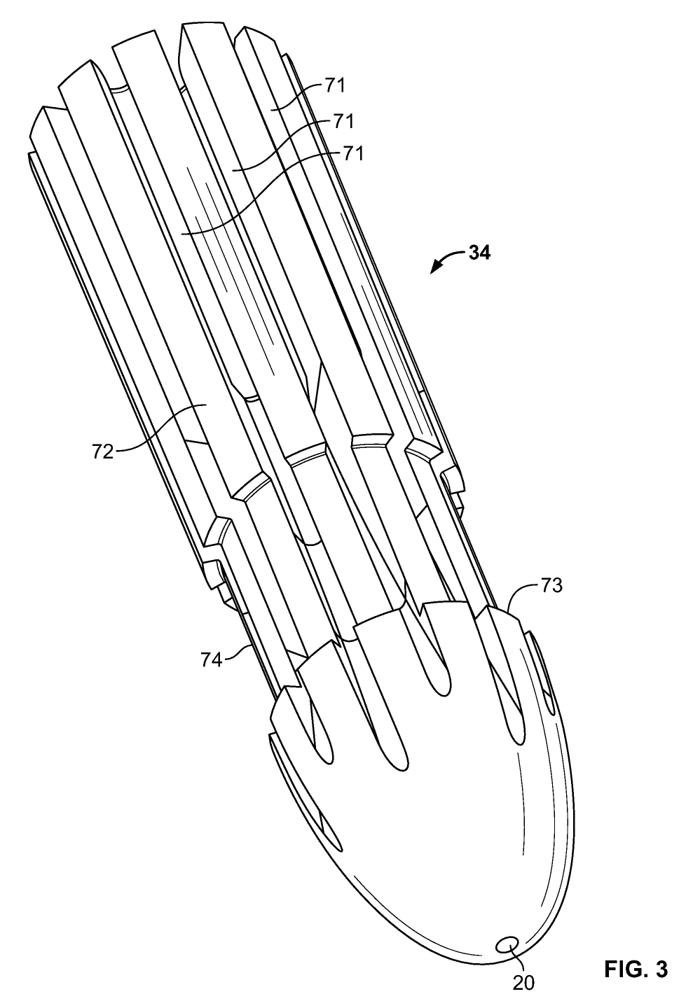

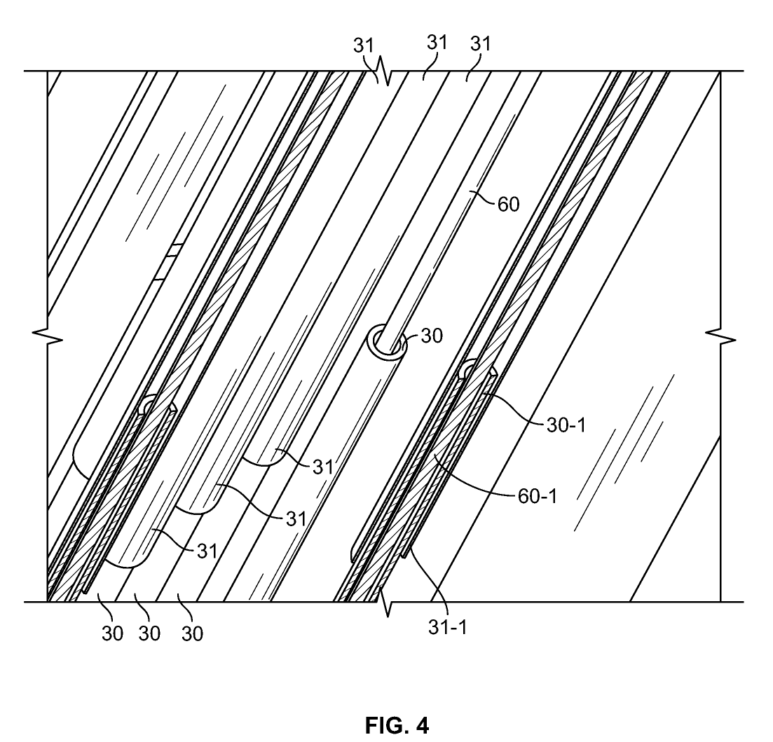

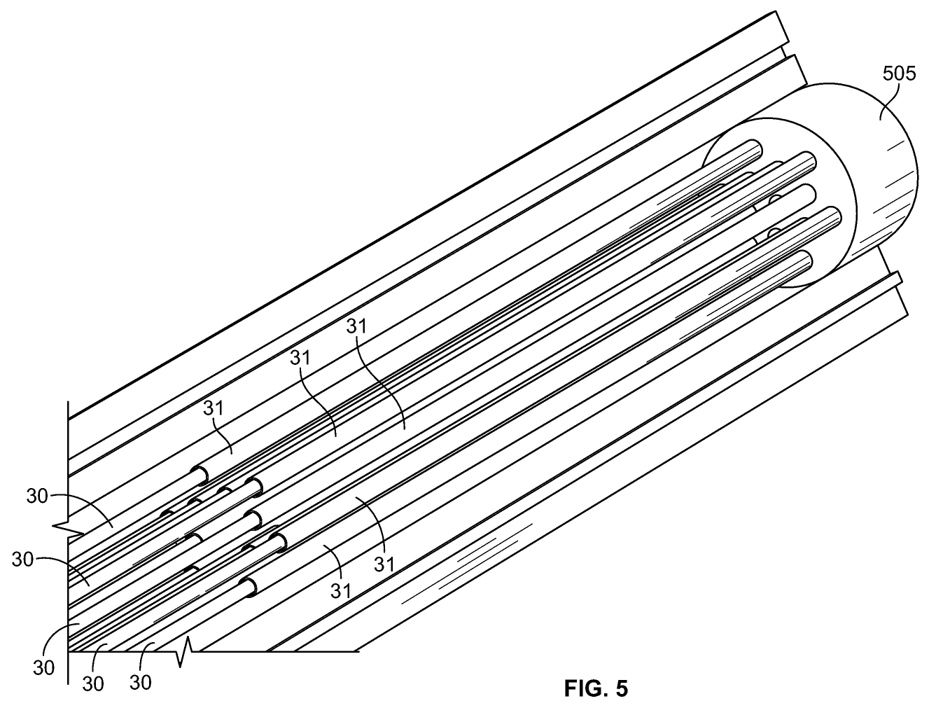

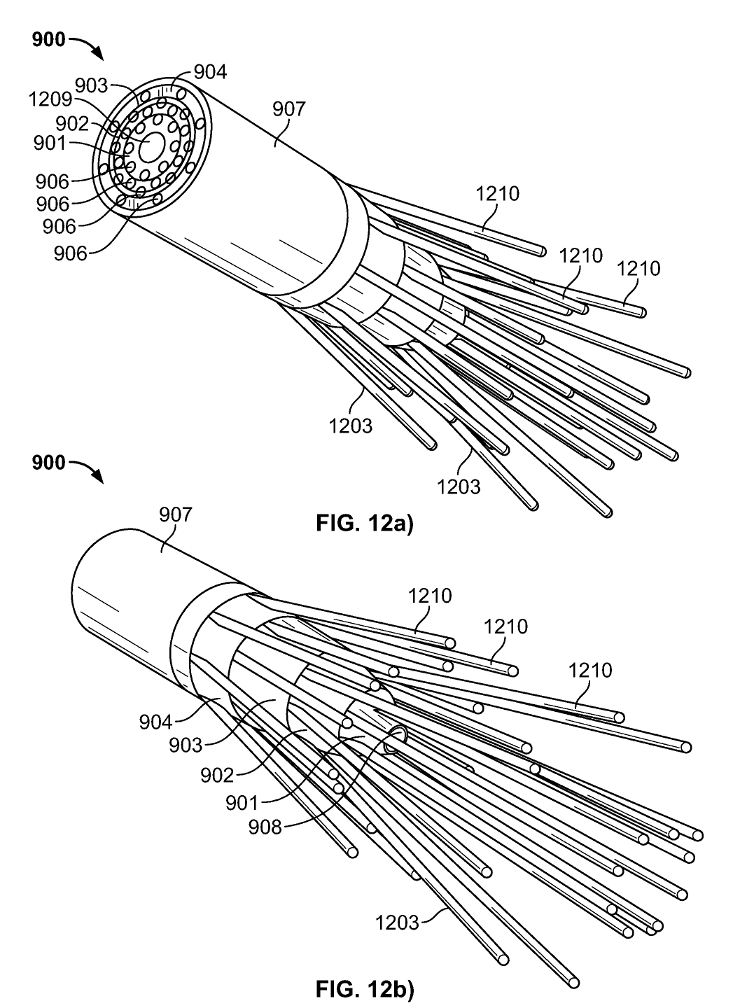

FIG. 1 together with FIG. 2 illustrate that the shaft 10 further comprises a plurality of electrode channels 50, and tip electrode channels 70 with respective outlets 71, distributed around the central access channel 20, and extending from the proximal end 12 to the distal end 11 of the shaft 10, and through the distal tip 13. Refer to FIG. 3 for a detailed view of the spatial arrangement of the tip electrode channels 70 in the distal tip 13. Each electrode channel 50 is adapted for guiding a single elongate electrode 60 that is slidingly movable relative to the shaft 10 between a first retracted position, and a second extended or advanced position (as shown in FIG. 12 for the second embodiment of the invention), where the electrodes project to an outside of the shaft 10. Each electrode 60 is enclosed in and slidably arranged along at least a section of the shaft 10 and along at least a section of the handle section 100, inside a corresponding electrode tube or cannula tube 30 of a first set of electrode tubes. Refer to FIGS. 4 and 5 for a detailed view of the electrode tubes 30. In the following, the expressions electrode tube and cannula tube are used interchangeably for the same feature.

Each electrode 60 comprises a proximal end 62, extending into the handle section 100, a distal electrode tip 61 and an intermediate region 63 electrically connecting the proximal end 62 and the distal electrode tip 61 of each electrode 60. The proximal ends 62 of the electrodes 60 are shaped to act as connectors, thus providing a means of electrically connecting the electrodes 60 to an electrical stimulus generator (not shown) that supplies a single electric pulse or a sequences of electric pulses according to preselected electroporation protocols for drug and gene delivery. The electric pulses have sufficient amplitude to generate an electric field in a target tissue that creates transient permeabilization of cell membranes or, alternatively, permanent permeabilization of cell membranes (irreversible) and/or an electrophoretic effect in the vicinity of the distal electrode tips 61 once the introducer device 1 is placed in or adjacent to a target tissue with the electrodes 60 extended to their advanced positions. This is explained in further detail in connection with the use of the electroporation device below.

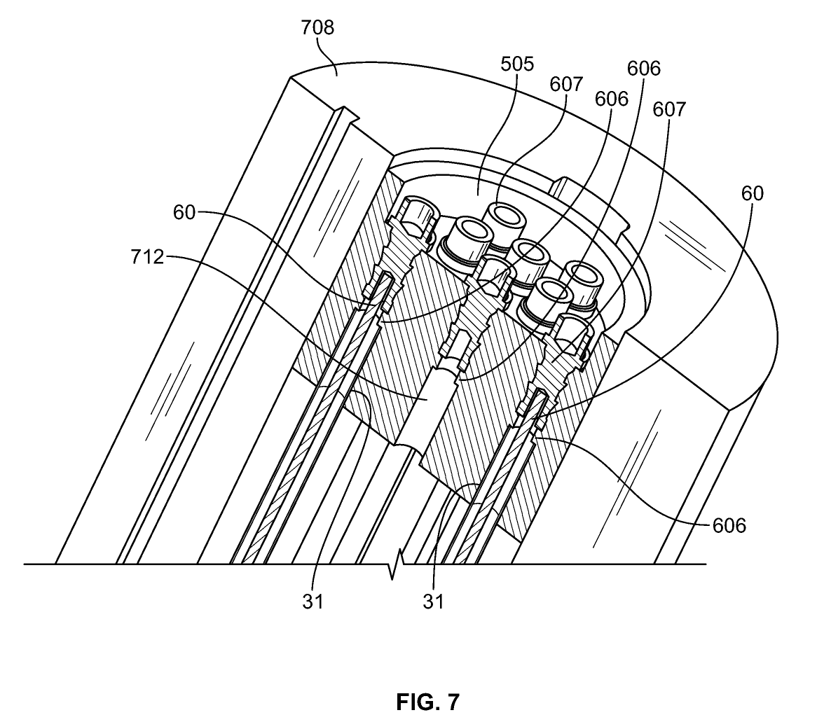

The proximal ends 62 of the electrodes 60 are fixedly attached to an adaptor plate 90 by means of respective connector shoes (not shown in this drawing) mounted in mating apertures of the adaptor plate 90. FIG. 7 illustrates the mounting of the connector shoes in the adaptor plate 90 and the mounting of the proximal ends 62 of the electrodes in greater detail. Reverting to FIG. 2, a second set of electrode tubes 31 are also fixedly attached in the adaptor plate 90 and extending along the longitudinal axis. Each of these electrode tubes 31 encloses a proximal end 62 of an electrode 60. By this arrangement, a plurality of pairs of coaxially arranged electrode tubes is accordingly formed from the first and the second sets of electrode tubes 30, 31. Each pair of electrode tubes is furthermore slidably arranged relatively to each other so as to form a telescopic arrangement. Refer to FIG. 4 for a detailed illustration and description of the plurality of telescopic arrangements.

The electroporation device 1 furthermore comprises a steering plate 91 or spacer which forms part of an electrode guide assembly mounted in a transition region between the shaft 10 and the handle section 100. The set of electrodes 60, electrode channels 50 and/or first electrode tubes 30 extend through respective holes in this steering plate 91 to keep the first or inner electrode tubes 30 locked in position inside an inner lumen of the shaft 10. The electrode distribution pattern and denseness may vary along a length dimension of the electroporation device 60 as illustrated in FIG. 2. The electrodes 60 may lie closer together in the distal tip 13 and in the shaft 10, than in the handle section 100 and the steering plate 91 may have been provided with an appropriate form and fit to accomplish this change in electrode denseness.

The intermediate regions 63 of the electrodes 60 are movably received in respective ones of the inner electrode tubes 30 arranged inside the electrode channels 50. In the present embodiment, each electrode is mounted inside a dedicated electrode tube 30 which supports, protects and electrically insulates the electrode in question from the other electrodes 60. The electrode tips 61 of the electrodes 60 are electrically conductive electrode segments that are movably received in the tip electrode channels 70 engraved into the outer contour of the single unitary rotationally symmetrical member 34 of the distal tip 13. Each tip electrode channel terminates in a forwardly pointing outlet 71 in the outer surface of the distal tip 13. Each tip electrode channel 70 further communicates with a corresponding electrode channel 50 in the shaft proper 10. Thus, movement of the electrodes 60 in a longitudinal direction (with respect to the longitudinal axis of the shaft 10) between a first retracted position, where the distally disposed electrode tips 61 are concealed within the distal tip 13, and a second advanced or extended position, where the electrode tips 61 project to an outside position of the distal tip 13, is allowed.

The shaft 10 may be entirely formed in, or comprise, a plastic or metallic material such as titanium, stainless steel or an injection moulded polymeric material. The outer diameter of the shaft is preferably five (5) millimetres or smaller, preferably between gauge 17 to 14 incl. The wall thickness of the shaft is preferably between 0.05 mm and 0.25 mm. The electrode channels 50, and tip electrode channels 70 may be formed in a suitable material, e.g. formed in a thermoplastic elastomer or a similar electrically insulating material. Each of the electrodes 60 is fabricated in, or at least comprises, an electrically conductive material such as 35 N LT metallic alloy, titanium, stainless steel or the like. The length of the shaft 10 is preferably between 50 mm and 500 mm depending on the particular application. As described above, the cross-sectional shape of each of the electrodes 60 is preferably essentially circular. However, in other embodiments, other cross sectional shapes may be applied. The diameter and cross-sectional shape of the tip electrode channels 70 are in any event preferably dimensioned for the desired electrode diameter and cross-sectional shape, in order to provide the best possible support for the electrodes, without impeding their ability to translate between their retracted position and extended positions. In the present embodiment of the invention, the electrode diameter is preferably 0.4 mm or smaller, such as smaller than 0.3 mm or 0.25 mm including a possible electrically insulating coating. The stiffness of an electrode is correlated to the cross-sectional area of the electrode such that thicker electrodes are stiffer. For some applications a stiff electrode may be necessary, e.g. if the target tissue is dense or hard. In soft tissue a less stiff electrode may be applied.

While positioned in the first retracted position, which is the default state of the device 1 outside the patient's body, the electrode tips 61 of the electrodes 60 are arrested inside the respective tip electrode channels 70, or alternatively even further retracted to a position wholly within the respective electrode channels 60, thus permitting the minimally invasive insertion of the electroporation device 1, i.e. causing minimal damage to surrounding tissue.

The tip electrode channels 70 are shaped with different curvatures to ensure deployment of the distal electrode tips 61 in a predetermined pattern where a largest distance between a pair of oppositely arranged electrode tips 61, measured in a plane transversal to the longitudinal axis of the introducer shaft, is larger than a largest cross-sectional dimension of the shaft 10 —in a plane perpendicularly to the longitudinal axis of the introducer shaft 10. Accordingly, the present electroporation device 1 makes it feasible to access deeper lying tissues, e.g. within the brain, trough a single tissue path using a single introducer shaft 10 with a distal tip 13 shaped to spread the intervening tissues during device insertion. Once the distal tip 13 reaches the treatment region, the electrodes 60 can be advanced to contact and/or enclose the target tissue. This allows an operator (surgeon) to treat a target tissue region or volume which has a cross-sectional dimension/extent much larger than the cross-sectional area of the introducer shaft 10. In order to provide the above described spatial distribution of the electrode tips 61, the tip electrode channels 70 are formed such that at least some of the tip electrode channels 70 curve outwardly, i.e. away from the longitudinal centre axis of the shaft 10 as previously mentioned.

The respective curvatures of the tip electrode channels 70 are preferably the sole mechanism for determining the travel trajectories of the distal electrode tips 61 during electrode advancement. Alternatively, the deflection away from the longitudinal axis of one or more electrodes 60 may be provided by other or additional deflection mechanisms e.g. a pre-tensioning or biasing of the electrodes in question. Such tensioning may be provided by a suitable choice of electrode materials, e.g. a shape memory alloy such as Nitinol, or by forming the (flexible) electrode e.g. in a bent shape, such that when it is arranged in a straight electrode channel 50 of the shaft 10 it is held in tension.

The electroporation device 1 illustrated FIGS. 1 and 2 comprises twelve electrode outlets 71 arranged in the distal tip 13 and associated with respective electrode channels 50 as depicted on FIG. 2. The electrode channels 71 of the distal electrode tip 13 are all formed in a single unitary rotationally symmetrical member 34 which minimizes any risk of detachment of individual tip parts. The second embodiment of a distal tip construction, as depicted on FIGS. 9-12, comprises eight tip electrode channels formed in each of the four coaxially arranged rotationally symmetrical members. However, an electroporation device according to the present invention may be provided with any practical number of electrodes 60 taking relevant size constraints of the device in question into consideration. The distribution of the electrode channels 50, and the associated set of electrodes 60, over a cross-section of shaft 10 illustrated by FIGS. 1-2, is such that the all electrodes 60 run substantially parallelly to the longitudinal axis of the shaft 10 with equidistant angular spacing around the longitudinal axis.

Each electrode 60 is formed in an electrically conductive material with each electrode 60 being arranged in an electrically insulating electrode channel 50, for example by choosing an electrically insulating material for the first set of electrode tubes 30. Alternatively and/or additionally, parts of the electrodes 60 may be coated with an electrically insulating layer or sheath, such that only the most distal tip portions 61 of the electrodes 60 are electrically conductive, i.e. un-insulated. Thus, the electric pulses will create an electric field spanning the distance from point to point (distal tip to distal tip), and a readily controllable firing pattern and thus a more controllable and accurate electric field may thus be generated by suitable selection and assignment of electrodes.

At least some subsets of the set of electrodes 60 is/are preferably of different length than other subsets of electrodes so that the respective electrode tips project to different positions along the longitudinal axis of the shaft 10 for example in a manner where the distal tips of each subset of electrode tips is positioned in a common plane perpendicular to the longitudinal axis of the shaft 10. Alternatively, all electrode tips may project to positions essentially lying in the same perpendicular plane along the longitudinal axis, L.

A deployment mechanism (not shown) acting on the electrodes 60 through the adaptor plate 90 may be manually driven or motorized (e.g. electronically controlled). The deployment mechanism may be adapted to advance all electrodes 60 simultaneously as a set, or individually, or in a number of subsets. The device 1 according to the invention may further be controlled by an electronic control unit (not shown), either incorporated in the device 1 or connectable to the device 1 through a cable or a wireless connection. In the wireless configuration, a suitable power supply is preferably located inside the device. The electronic control unit may be programmable, such that a desired electrode stimulus pattern may be programmed prior to a treatment session or procedure.

In alternative embodiments (not shown), and as mentioned above, a partially disposable device variation of the above described embodiments is proposed, with a disposable introducer shaft 10 and non-disposable (re-usable) handle section 100 comprising a deployment mechanism with interfaces to electrodes formed in the disposable introducer shaft 10 and an electrical connections that may be customized to individual electrical stimulus generators.

During patient treatment or in use, the connector plug of the electroporation device ensures the electrodes 60 are electrically connected to a suitable electrical stimulus generator. The shaft is subsequently inserted through a bore hole in the patient's skull and advanced to the target region or tissue inside the patient's body/brain. The precise location may be identified by means of ultrasound, CT, MR or another suitable means, and the correct position of the introducer shaft 10 prior to deployment may be verified by similar means. As described above, in other embodiments, the stimulus generator may be integrated in the handle section 100. When the introducer shaft 10 has been appropriately located, a doctor may deliver a suitable chemotherapeutic agent or dose of genetic material through the central channel 20 and into the tissue region to be treated. Delivery is done by inserting the elongate, length-adjusted and properly dulled needle of a syringe (not shown) in the central hole and advancing it until no further motion is possible. The operator may then empty the syringe barrel by pressing the syringe plunger, whereupon the liquid content of the syringe is transported into the tissue to be treated. Prior, during, or upon delivery, the doctor may deploy or advance the electrodes 60 to form a predefined electrode tip pattern. In one particular preferred embodiment the un-insulated electrode tips 61 are positional and positioned in a manner where they at least partially surround or enclose the target tissue in such a way that the distal tips 61 describe or define an outer periphery of a spherical/spatial ellipse. After electrode deployment, the doctor activates the electric stimulus generator to deliver a sequence of preferably short and intense electric pulses, for example square-wave pulses, to the tissue to be treated. To ensure a suitable distribution of pulses with associated electrical fields penetrating the target tissue, pulses may be assigned to alternating specific electrodes in a pattern that is tailored to the anatomy of the individual region of the body to be treated and/or the geometry of specific malignant target tissue. In an embodiment, at least some of the end-points 61 of the electrodes 60 are placed in equidistant relation to other electrode end points 61, and at least some pulses are assigned to equidistant pairs of electrodes. Thus, a homogenous or heterogeneous, controllable three-dimensional electric field can be created in the target tissue.

Upon pulse delivery, the doctor may retract the electrodes 60 to their first retracted position by actuating the deployment mechanism such that the electrodes are retracted to their default position within the distal tip 13, and the device 1 may be removed from the body of the patient. Alternatively, the doctor may reposition the electroporation device after having retracted the electrodes 60, potentially permitting multiple pulse applications covering a larger area in a single device insertion. A saline solution may be injected into the target tissue through the access channel 20 to enhance the electroporation process by increasing tissue conductivity. In either case suitable means for connecting the channel 20 to a source of saline solution may preferably be provided at the handle section. 100

The electrodes may also be prepared with/covered by/impregnated with a drug or DNA molecule compound that may be dissolvable in an electrical field. Thereby, a drug etc. may be released from the electrodes when an electrical field is applied to the target tissue via the electrodes. Thereby the access channel 20 may be spared. However, the drug impregnated electrodes may also be used with embodiments having a delivery channel 20 in order to release multiple drugs or in order to save the delivery channel for e.g. a field enhancing saline solution as described above.

FIG. 3 is a perspective view of the single unitary rotationally symmetrical member 34 of the distal tip 13 of the electroporation device 1 described above in connection with FIG. 1 and FIG. 2. The distal tip 13 is detachably mounted to the shaft 10 of the electroporation device 1 by a locking mechanism described below. The single unitary rotationally symmetrical member 34 or unitary member comprises 12 longitudinally extending tip electrode channels 71 formed as respective electrode grooves 71 engraved into the circumferential contour or outer wall 72 of the unitary member 34. The electrode grooves 71 extend in a substantially parallel manner along the longitudinal axis of the electroporation device and are arranged at different depths below the circumferential contour 72. Together the grooves 71 form an intermittent pattern of relatively deeply embedded grooves and relatively shallowly arranged grooves. A circumferential collar 73 is formed by a radial cut-out 74 in the circumferential contour 72 of the single unitary rotationally symmetrical member 34. The circumferential collar 73 contacts and abuts a mating distal annular edge 33 of the shaft 10 when the distal tip is appropriately positioned inside an inner lumen of the shaft 10 as illustrated on FIG. 3a and described below.

FIG. 3a) illustrates how the distal tip 13, including the single unitary rotationally symmetrical member 34, and the shaft 10 are coupled and mutually locked. The shaft 10 comprises a plurality locking apertures 37 and the distal annular edge 33. The distal tip 13 comprises three essential identically sized semi-cylindrical shells 35 that are placed in mutual abutment inside the radial cut-out 74 of the single unitary rotationally symmetrical member 34 (see FIG. 3) so as to surround this portion of the single unitary rotationally symmetrical member 34. Each of the semi-cylindrical shells 35 comprises a toothed distal edge where individual teeth 39 frictionally engage and lock to a corresponding electrode groove 71. The teeth additionally function as support and guiding mechanisms for the individual electrodes 60 forcing each of these toward a bottom wall of electrode groove 71. One or both outer surfaces of the semi-cylindrical shells 35 comprises a snap lock protrusion 36 mating to a locking aperture 37 arranged in a wall section of the shaft 10. When the distal tip 13 is pressed or slid into the inner lumen of the shaft 10 in the direction indicated by arrow 38 the snap lock protrusion 36 engages with the mating locking aperture 37. At the same time, the circumferential collar 73 of the single unitary rotationally symmetrical member 34 is brought into contact with the distal annular edge 33 of the shaft 10 to form a smooth non-cutting transition region or area between these individual parts. The mating and cooperating snap lock protrusion 36 and locking aperture 37 serve to lock or fix the distal tip 13 to the shaft 10 in a direction along the longitudinal axis and rotationally too so as to ensure these parts are firmly interconnected during treatment procedures.

FIG. 4 is a close-up view of the plurality of telescopic arrangements formed between the first and second sets of electrode tubes 30, 31, respectively. A round electrode 60-1 is enclosed within its associated inner electrode tube 30-1 and an outer electrode tube 31-1 surrounds the inner electrode tube 30-1. Both the inner and outer electrode tubes 30-1 and 31-1, respectively, are shown in sectional view. In a telescopic arrangement a cooperating pair of the electrode tubes e.g. 30-1 and 31-1 is arranged co-axially with one electrode tube on the outside sliding over and around an inner electrode tube in connection with actuation of the deployment mechanism (not shown). During electrode advancement the round electrodes 60 are displaced to the advanced position where the distal electrically conductive electrode tips project to the outside of the distal tip. When the electrodes are extended to their advanced positions, the overlap distance between each telescopic pair of inner and outer electrode tubes is larger than the corresponding overlap distance when the pair of electrode tubes is positioned in the retracted position. The overlap distance of a pair of electrode tubes of each telescopic arrangement may be adapted to dimensions of a specific electroporation device, but may be larger than 20 mm, such as between 30 mm and 100 mm, measured along the longitudinal axis in the advanced position of the electrodes and about 2-10 mm in the retracted position. It is understood that all electrodes 60 preferably are arranged inside a respective one of the electrode tubes 30. Alternatively, more than one of the inner electrode tubes 30, such as for example to, three, four or five inner electrode tubes, may be arranged inside a single one of the outer electrode tubes 31 in a single telescopic arrangement. Furthermore, it is contemplated that more than a single round electrode 60, e.g. such as for example two, three, four or five round electrodes may be arranged inside a single inner electrode tube 30. Likewise, it is understood that each of the outer electrode tubes 31, which are fixedly attached to the adaptor plate, in the alternative may be arranged inside the electrode tubes 30 instead of the outside arrangement depicted in FIG. 4.

FIG. 5 shows a cross-sectional view of the electroporation device 1 with the outer or second set of electrode tubes 31 coupled to an adaptor plate 505. The first or inner electrode tubes 30 are arranged inside respective ones of the outer electrode tubes 31 and form the previously described plurality of telescopic arrangements. The outer electrode tubes 31 are attached in respective appropriately dimensioned apertures of the adaptor plate 505. The adaptor plate 505 is displaceable in order for displacing the outer electrode tubes 31 and the electrodes 60 (refer to FIG. 4) which are slidably arranged inside respective ones of the inner electrode tubes 30.

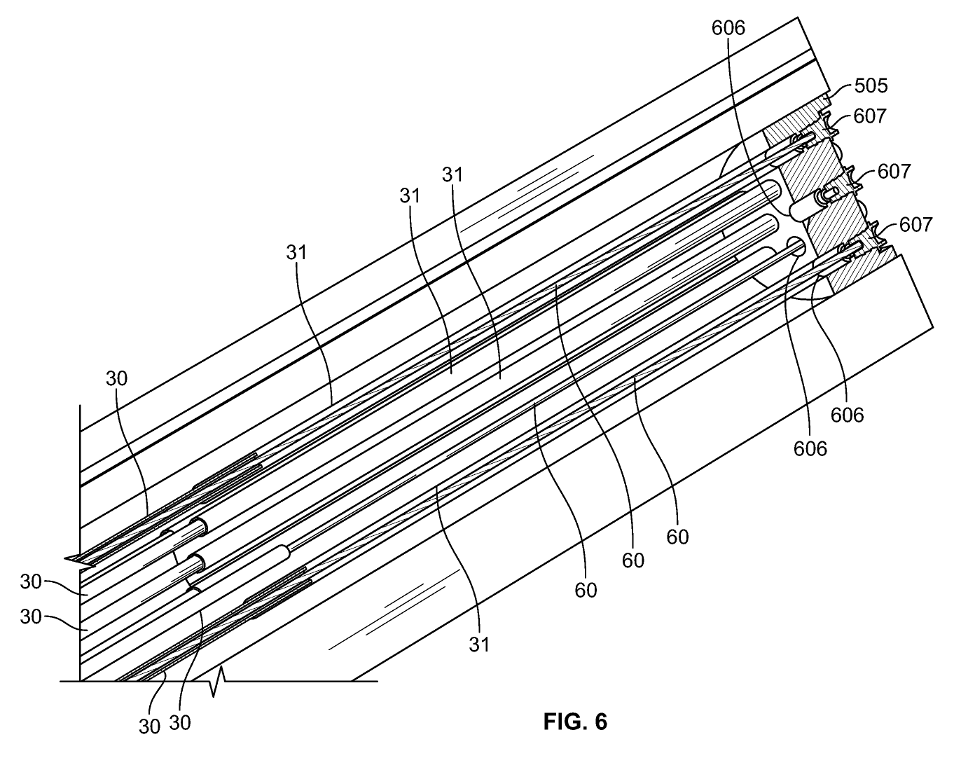

FIG. 6 is a cross-sectional view similar to FIG. 5 but with the adaptor plate 505 depicted in a cross-sectional view revealing internal construction details. The adaptor plate 505 comprises 12 mounting apertures 606 each having a mating connector shoe 607 inserted therein. Furthermore, a proximal end of each of the outer electrode tubes 31 is inserted into a respective one of the connector shoes 607.

FIG. 7 is a close-up and radial cross-sectional view of the adaptor plate 505 with mounted connector shoes 607. The adaptor plate 505 is inserted into a circular aperture arranged centrally in a rear portion 708 of the handle section 100. Each connector shoe 607 is inserted into a corresponding mounting aperture 606 of the adaptor plate 505 and rigidly connected to the adaptor plate 505 by an appropriate attachment mechanism such as soldering, welding, adhesively or press-fitting etc. The proximal ends of the outer electrode tubes 31 project into the respective mounting apertures 606 from an opposing side of the adaptor plate 505 and encloses proximal end sections of the electrodes 60 to support and guide these and protect them against buckling.

Each connector shoe 607 comprises an annular collar adapted to rest on an adaptor plate surface. When a connector shoe 607 is inserted into the corresponding mounting aperture 406, the connector shoe is advanced into mounting aperture 406 until the annular collar 603 rests on the adaptor plate surface. A number of barbed outer ribs are shaped so that they allow the connector shoe 607 to be pressed into the adaptor plate 505 from above but the barbs prevent the connector shoe 607 from being released again. The proximal end of an electrode is attached in one end of the connector shoe 607. The proximal end of the electrode is squeezed into and soldered to the distal end of the connector shoe 607 to establish reliable electrical interconnection. The other end of the connector shoe 607 is electrically connected to a flexible lead or cable (not shown) leading to the electrical stimulus generator or pulse generator.

In the present embodiment of the invention, a length of the entire connector shoe 607 may be between 3.0 and 4.0 mm such about 3.6 mm. A diameter of the proximal end may be about 1.2 mm and a diameter of the distal end may be about 1 mm. The connector shoes comprise an electrically conductive material such as a metal or an alloy.

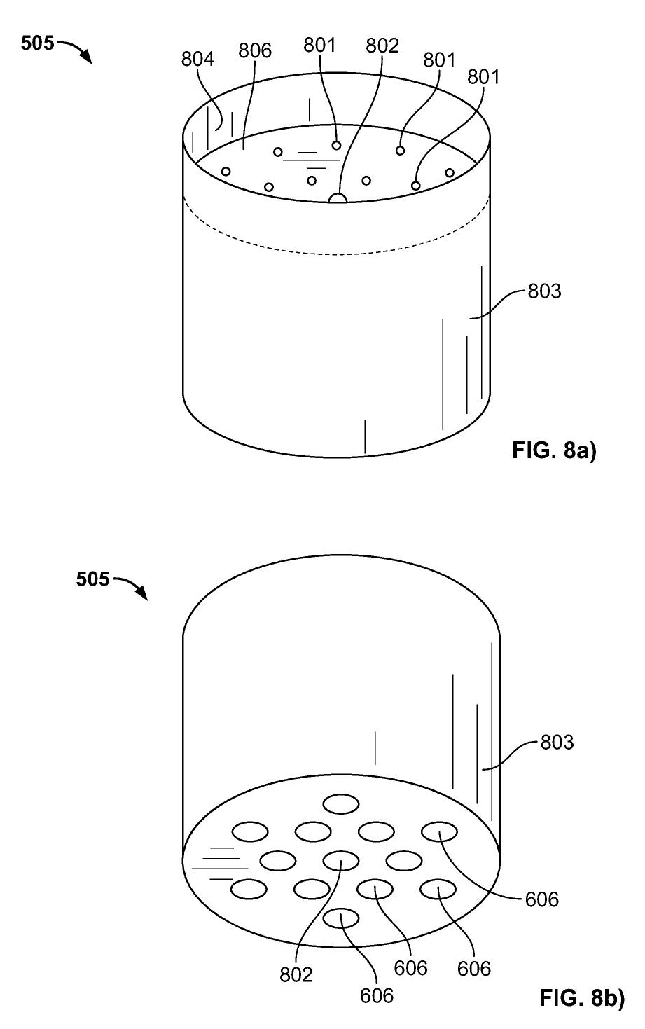

FIGS. 8a) and b) are two different perspective views of the adaptor plate 505 in an unassembled state. FIG. 8 a) illustrates the adaptor plate 505 seen from the distal end of the electroporation device and FIG. 8 b) shows the adaptor plate 505 seen from the proximal end of the electroporation device. The adaptor plate 505 comprises a central hole or bore 802 for holding a connector shoe or a tube to create a fluid delivery channel or central electrode (not shown). The adaptor plate structure comprises an outer cylindrical wall 803 and an internal cylindrical wall 804. A plane face 806 is lowered into the adaptor plate 505 to create a basin 806 enclosed by the adaptor plate 505 and the cylindrical wall 504. When the connector shoes are inserted into the mounting apertures 801, the basin 806 is preferably filled with an insulating and/or adhesive agent or material (not shown) for the purpose of mutually insulating proximal ends of the electrodes and from an external environment. The adhesive agent or material furthermore ensures an improved attachment between the connector shoes and the electrode proximal ends. The adaptor plate 505 can be made of any suitable material for an electrically insulating material for preventing formation of short circuits between electrodes. The adaptor plate material can be a polymeric material, a ceramic material, a printed circuit board made of e.g. woven glass, cotton paper, epoxy and/or polyesther and/or the like.

As previously mentioned, each proximal electrode end is inserted into a connector shoe 607 which in turn is inserted into a mounting aperture 606 of the adaptor plate 505. The mounting apertures 606 are placed in pattern resembling that of the electrodes when they emerge from the outer electrode tubes (30 on FIG. 7) and/or from an electrode guide assembly (not shown). The pattern and the denseness of the electrodes may not be the same along the entire length of the electroporation device, since the electrodes may lie closer together in the distal tip and in the shaft, than in the handle section. Thus the pattern and the denseness of the electrodes may be widened at a point where the shaft and the handle section meet.

FIG. 8b) shows the adaptor plate 505 in the unassembled state from the proximal end of the electroporation device. The proximal end of the adaptor plate 505 is configured for attachment of the electrodes as indicated with thin vertical lines projecting into the mounting apertures or holes 606 and further into the respective connector shoe.

FIGS. 9 a) and b) are frontal and cross-sectional views, respectively, of a multi-element detachable distal tip 900 coupled to a distal portion of an elongate introducer shaft 907 according to a second embodiment of the invention. The present multi-element distal tip 900 or distal tip is suitable for an alternative electroporation device in accordance with a second embodiment of the invention which comprises a larger number of electrodes than the first embodiment described above. The distal tip 900 comprises four rotationally symmetrical tip members 901, 902, 903, 904 formed as respective hollow shells, where each hollow shell comprises a set of eight electrode channels formed by longitudinally extending electrode grooves 906 around an outer peripheral contour or wall of each hollow shell. An electrode channel is accordingly formed by cooperation between a electrode groove and inner wall section of the coaxially arranged surrounding outer hollow shell. Each of hollow shells 901, 902, 903 and 904 is rotationally symmetrical about the longitudinally axis, L, i.e. a tip member looks the same after a fixed amount of rotation. In the present case each hollow shell has 8-fold rotational symmetry.

An innermost hollow shell 901 is surrounded, enclosed, and encased by a coaxially arranged second hollow shell 902, which itself is surrounded, enclosed, and encased by third hollow shell 903, which itself is surrounded, enclosed, and encased by a fourth, outermost, hollow shell 904 forming a tip construction where all hollow shells 901-904 are arranged coaxially relative to each other. Each hollow shell comprises eight electrode channels formed by respective grooves 906 engraved into the respective outer contours of the hollow shells and terminating in respective electrode outlets 905. The electrode channels or electrode grooves 906 are adapted to enclose and guide respective ones of a set of 32 electrodes. When the electrodes are placed in their advanced positions, distal electrical conductive or uninsulated electrode tips (items 1210 of FIG. 12) project outside of the distal tip 900 through corresponding tip electrode channel outlets as illustrated in FIGS. 12 a) and b).

A central access channel 909 extends centrally through the innermost hollow shell 901 and terminates in a frontal portion thereof in form of outlet 908. The arrangement of the access channel 909 is illustrated in detail in the cross-sectional view of FIG. 9b).

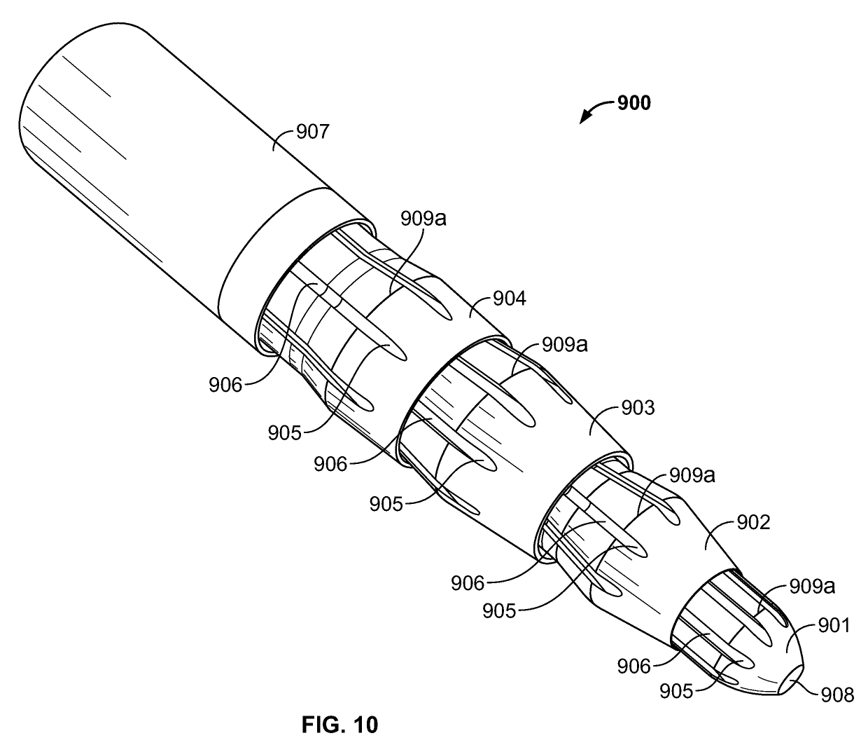

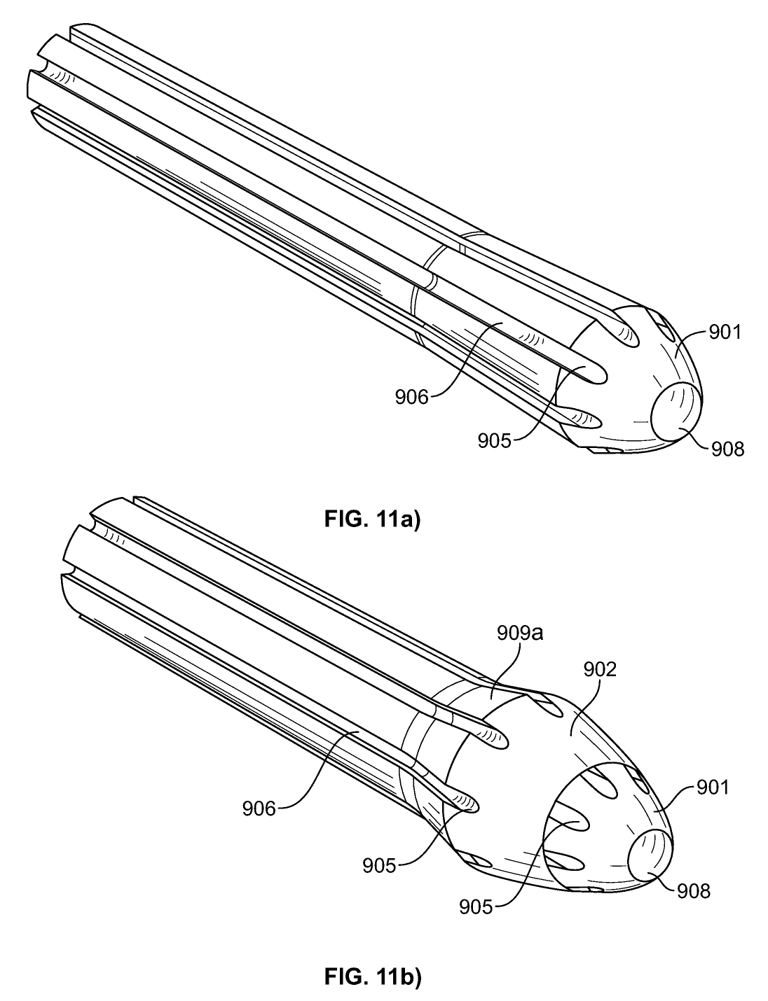

FIG. 10 illustrates the four hollow shells 901, 902, 903 and 904 of the multi-element distal tip 900 in a partially disassembled state of the distal tip 900. The rear portion of outermost hollow shell 904 is inserted into, and press-fitted against, a mating cylindrical inner wall section of a distant portion of an elongate introducer shaft 907. Each of the hollow shell preferably comprises a circumferential collar or rise 909a. The circumferential collars 909a function as respective mechanical locking mechanisms for the four hollow shells 901, 902, 903 and 904. The circumferential collars 909a prevent forward movement of the hollow shells along the longitudinal axis away from the inner lumen of a surrounding, outer, hollow shell. Furthermore, the positions of the circumferential collars 909a align relative positions of the hollow shells 901, 902, 903 and 904 to each other in longitudinal direction to ensure appropriate positioning of the electrode outlets 905. The circumferential collar 909a of the outer hollow shell 904 likewise locks the hollow shell 904 to the shaft 907 thus ensuring that the entire tip assembly 900 is appropriately locked to the electroporation device.

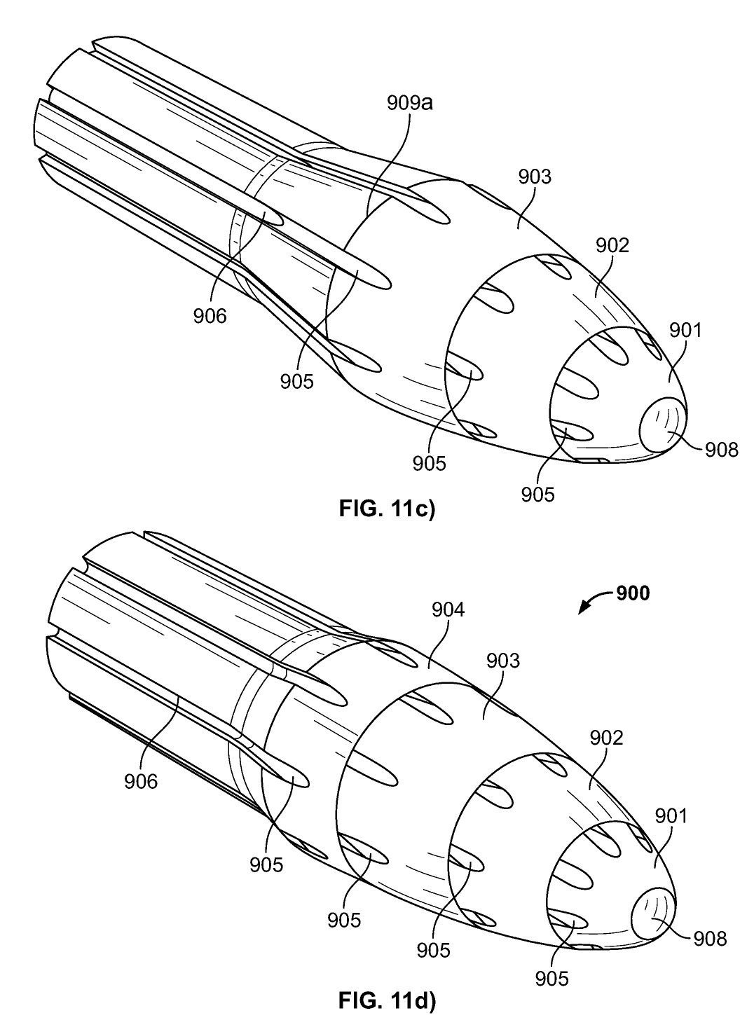

FIG. 11 a)-d) is a series of illustrations of construction details of the multi-element distal tip 900 in various states of assembly. In FIG. 11a) the first, or inner-most, hollow shell 901 is depicted alone. The electrode outlets 905 of the tip electrode channels formed by grooves 906 in the hollow shell are readily visible. In FIG. 11b) the first hollow shell 901 and the second hollow shell 902 are depicted in an assembled and interlocked state. FIG. 11c) depicts the first hollow shell 901, the second hollow shell 902 and the third hollow shell 903 in an assembled and interlocked assembly state. The plurality of electrode outlets 905 in each hollow shell is depicted. FIG. 11d) shows a complete distal tip 900 wherein the first hollow shell 901, the second hollow shell 902, the third hollow shell 903 and the fourth hollow shell 204 are interlocked and assembled to a multi-element structure with a plurality of the tip electrode channels disposed in four radially displaced rings or circles to provide a unique spatial coordinate of each of the electrodes in its advanced position.

Each of the grooves 906 in a hollow shell in engraved lower down in a proximal end of the hollow shell than in its distal end. This provides curvature away from the longitudinal axis of each groove so the electrodes instead of advancing essentially parellelly to the longitudinal axis out of the respective electrode outlets 905 are bent sidewards for spreading the spatial positions of electrode tips. Each of the grooves, or each set of grooves in a specific hollow shell, may of course be provided with an individual shape/deflection/curving in order to provide a specific pattern or distribution of the electrode tips in their advanced positions.

FIGS. 12 a) and b) is rear and frontal views, respectively, of the distal tip 900 with an exemplary set of 32 electrodes 1203 slidably arranged inside respective tip electrode channels 906. Each electrode 1203 terminates in an uninsulated distal electrode tip 1210 that project to a position beyond the distal tip 900 along the longitudinal axis. The 32 electrodes are all placed in an advanced position. While the present multi-element distal tip 900 comprises four hollow shells 901, 902, 903 and 904 other embodiments may comprise fewer or additional hollow shells depending on a specific electroporation application and its accompanying constraints on device dimensions and number of electrodes.

1. An electroporation device, comprising:
a handle section operatively connected to an elongate introducer shaft,
the elongate introducer shaft having a longitudinal axis and a distal tip,
a set of electrodes comprising respective distal electrode tips,
an electrode guide assembly arranged inside the elongate introducer shaft and comprising a set of electrode channels with each electrode channel guiding one or several electrodes,
each electrode being slidably arranged within an electrode channel between a retracted position, where the distal electrode tips are enclosed inside the introducer shaft, and an advanced position, where the distal electrode tips project to an outside of the introducer shaft,
a set of longitudinally extending tip electrode channels arranged in the distal tip and adapted to guide respective travel trajectories of the distal electrode tips during electrode advancement and retraction; wherein
the set of longitudinally extending tip electrode channels comprises a set of electrode grooves disposed around a circumferential contour of a rotationally symmetrical member of the distal tip.

2. An electroporation device according to claim 1, wherein the rotationally symmetrical member has n-fold rotational symmetry;
n being a positive integer number between 2 and 32, preferably between 4 and 16 such as between 6 and 12.

3. An electroporation device according to claim 1, wherein the set of electrode grooves is formed in a single unitary rotationally symmetrical member.

4. An electroporation device according to claim 1, wherein the set of electrode grooves comprises at least two grooves arranged at different depths in the circumferential contour of the rotationally symmetrical member.

5. An electroporation device according to claim 1, wherein the distal tip comprises a plurality of coaxially arranged rotationally symmetrical members;
the set of longitudinally extending tip electrode channels being formed as respective sets of electrode grooves engraved into respective circumferential contours of the plurality of coaxially arranged rotationally symmetrical members.

6. An electroporation device according to claim 1, wherein the distal tip comprises a pair of coaxially arranged hollow shells;
an inner, rotationally symmetrical, shell and an outer shell,
wherein the set of longitudinally extending tip electrode channels is formed by respective electrode grooves engraved into a circumferential outer wall of the inner hollow shell and inner wall sections of the outer hollow shell.

7. An electroporation device according to claim 1, wherein the distal electrode tips extend beyond the distal tip in the advanced position of the set of electrodes.

8. An electroporation device according to claim 1, wherein the distal electrode tips are deflectable away from the longitudinal axis of the elongate introducer shaft during electrode advancement to their advanced position such that at least one planar projection taken in a plane perpendicular to said longitudinal axis of a distance (D1) between at least one pair of electrode tips is larger than a maximum dimension of a cross-section of the elongate introducer shaft,
said cross-section taken in a plane perpendicular to said longitudinal axis at a distal end of the elongate introducer shaft.

9. An electroporation device according to claim 8, wherein the deflection of each of said distal electrode tips during electrode advancement is provided by a corresponding curvature of the longitudinally extending tip electrode channel.

10. An electroporation device according to claim 9, wherein the respective curvatures of the longitudinally extending tip electrode channels constitute a sole mechanism for determining the travel trajectories of the distal electrode tips during electrode advancement.

11. An electroporation device according to claim 1, wherein an outer contour of the distal tip comprises a substantially smooth, rounded, non-cutting outer contour with a substantially smooth, non-cutting transition to the elongate introducer shaft.

12. An electroporation device according to claim 1, wherein each of the electrode channels comprises a tube such as a metallic or plastic cannula tube.

13. An electroporation device according to claim 1, wherein each of the electrode channels within the electrode guide assembly is aligned with a corresponding longitudinally extending tip electrode channel of the distal tip.

14. An electroporation device according to claim 13, wherein the electrode guide assembly comprises a disc shaped or annular structure with through going openings or channels forming part of the electrode channels.

15. An electroporation device according to claim 1, wherein the elongate introducer shaft and the distal tip comprise an access channel extending there through along the longitudinal axis.

16. An electroporation device according to claim 15, wherein the set of electrodes comprises a tubular electrode mounted in the access channel.

17. An electroporation device according to claim 1, wherein a maximum dimension of a cross section of the elongate introducer shaft is smaller than 30 mm, preferably smaller than 10 mm, or more preferably less than 5 mm.

18. An electroporation device according to claim 1, wherein a cross section of the elongate introducer shaft is essentially circular, elliptical or polygonal.

19. An electroporation device according to claim 1, wherein the set of electrode channels comprise a first set of cannula tubes fixedly attached to the electrode guide assembly.

20. An electroporation device according to claim 19, further comprising:
a deployment mechanism operatively connected to the set of electrodes through an adaptor plate to displace the set of electrodes between the retracted and advanced positions,
a second set of cannula tubes fixedly attached to the adaptor plate;
the first and second set of cannula tubes forming a plurality of telescopic arrangements.

21. An electroporation device, comprising:
a handle section operatively connected to an elongate introducer shaft,
the elongate introducer shaft having longitudinal axis and a distal tip,
a set of electrodes at least partly enclosed in, and slidably arranged in, respective ones of a first set of electrode tubes and comprising respective distal electrode tips,
a deployment mechanism operatively connected to the set of electrodes to displace these between a retracted position, where the distal electrode tips are enclosed inside the introducer shaft, and an advanced position where the distal electrode tips project to an outside of the introducer shaft,
an electrode guide assembly arranged inside the elongate introducer shaft and comprising a set of electrode channels each guiding one or several electrode tubes of the first set of electrode tubes,
a set of longitudinally extending tip electrode channels arranged in the distal tip and adapted to guide respective travel trajectories of the distal electrode tips during electrode advancement and retraction; wherein
the deployment mechanism comprises an adaptor plate fixedly attached to the set of electrodes and to a second set of electrode tubes;
the first and second set of electrode tubes forming a plurality of telescopic arrangements.

22. An electroporation device according to claim 21, wherein each electrode tube of the second set of electrode tubes is substantially coaxially arranged relative to a respective one of the first set of electrode tubes.

23. An electroporation device according to claim 21, wherein each electrode tube of the first set of electrode tubes is arranged inside respective ones of the second set of electrode tubes.

24. An electroporation device according to claim 21, wherein each electrode tube of the first set of electrode tubes is arranged outside respective ones of the second set of electrode tubes.

25. An electroporation device according to claim 21, wherein each electrode tube of the first set of electrode tubes is arranged outside or inside a single electrode tube of the second set of electrode tubes.

26. An electroporation device according to claim 21, wherein the adaptor plate comprises a plurality of through-going mounting holes receiving respective electrodes and electrode tubes of the second set of electrode tubes.

27. An electroporation device according to claim 21, wherein respective proximal ends of the electrodes are inserted into the through-going mounting holes.

28. An electroporation device according to claim 21, wherein an overlap of each telescopic arrangement between the first and second set of electrode tubes is larger than 20 mm, such as between 30 mm and 100 mm, measured along the longitudinal axis in the advanced position of the set of electrodes.

29. An electroporation device according to claim 28, wherein the overlap of each telescopic arrangement between the first and second set of electrode tubes is larger than 2 mm, preferably larger than 5 mm, in the retracted position of the set of electrodes.

30. An electroporation device according to claim 21, wherein the distal tip comprises a rotationally symmetrical member wherein the set of longitudinally extending tip electrode channels comprises a set of electrode grooves disposed around a circumferential contour of the rotationally symmetrical member.

31. An electroporation device according to claim 1 or 21, wherein each of the distal electrode tips comprises an electrically un-insulated portion with a length between 10 mm and 50 mm.

32. An electroporation device, comprising:
a handle section operatively connected to an elongate introducer shaft,
the elongate introducer shaft having a longitudinal axis and a detachable distal tip,
a set of electrodes comprising respective distal electrode tips,
an electrode guide assembly arranged inside the elongate introducer shaft and comprising a set of electrode channels with each electrode channel guiding one or several electrodes,
each electrode being slidably arranged within an electrode channel between a retracted position, where the distal electrode tips are enclosed inside the introducer shaft, and an advanced position, where the distal electrode tips project to an outside of the introducer shaft,
a set of longitudinally extending tip electrode channels arranged in the detachable distal tip and adapted to guide respective travel trajectories of the distal electrode tips during electrode advancement and retraction; wherein
the detachable distal tip comprises a first locking member engaging a second locking member arranged on the elongate introducer shaft.

33. An electroporation device according to claim 32, wherein the first locking member comprises an annular protrusion or collar integrally formed with the distal tip and protruding from an outer surface contour thereof.

34. An electroporation device according to claim 32, wherein the distal tip comprises a plurality of coaxially arranged rotationally symmetrical members;
the set of longitudinally extending tip electrode channels being formed as respective sets of electrode grooves engraved into respective circumferential contours of the plurality of coaxially arranged rotationally symmetrical members.

35. An electroporation device according to claim 32, wherein:
the first locking member comprises a semi-cylindrical shell coupled to the distal tip and having a radially projecting pin,
the second locking member, arranged in a wall of the elongate introducer shaft, comprising an aperture or opening mating to the radially projecting pin.

36. An electroporation device according to claim 35, wherein the semi-cylindrical shell comprises a toothed distal edge with a plurality of individual teeth adapted to frictionally engage and lock to respective tip electrode channels of the set of longitudinally extending tip electrode channels of the detachable distal tip.

37. An electroporation device according to claim 32, wherein the set of longitudinally extending tip electrode channels are formed in a single rotationally symmetrical member of the distal tip.
